(19) United States
(12) Patent Application Publication
(10) Pub. No.: US 2003/0165994 A1
(43) Pub. Date: Sep. 4, 2003

(54) Device and method for multiparallel synthesis and screening

(76) Inventors: Sergey A. Kozmin, Chicago, IL (US); Ying Wang, Chicago, IL (US)

Correspondence Address: BRINKS HOFER GILSON & LIONE, P.O. BOX 10395, CHICAGO, IL, 60610, US

(21) Appl. No.: 10/172,337

(22) Filed: Jun. 13, 2002

Related U.S. Application Data
(60) Provisional application No. 60/361,827, filed on Mar. 4, 2002.

(51) Int. Cl. C12Q 1/00; G01N 33/53; B32B 5/02

(52) U.S. Cl. 435/7.1; 422/129

(57) ABSTRACT

A device and method for a multiparallel synthesis and screening using a three-dimensional array comprising a plurality of reaction wells having a means for delivery of a fluid or solid along three directions corresponding to the x, y, and z axes of the array. The invention makes use of a plurality of removable sealing elements each of which is positioned across or spans two sides of a reaction well when in a sealing position. The reaction wells may be made of materials such as polypropylene. The sealing elements may be in the form of cylinders, tubes, rods, or tapered pins. The amount of a reaction product produced in the synthesis can be as much as 5 mg or more.

### REFERENCE TO RELATED APPLICATIONS

This application claims the benefit of a U.S. Provisional Application No. 60/361,827 filed on Mar. 4, 2002, which is incorporated herein.

### FIELD OF THE INVENTION

The invention generally relates to a device and method for a multiparallel synthesis and screening. In particular, the device and method of the invention relates to a multiparallel synthesis and screening using three-dimensional arrays having a means for delivery of reagents along the x, y, and z axes of the array.

### BACKGROUND OF THE INVENTION

The ability to rapidly generate and screen new collections of small molecules is crucial to the efficiency of the development of new therapeutic compounds. Over the past decade, combinatorial chemistry has emerged as an alternative approach for new lead discovery and optimization. Initially, the focus was on the synthesis of peptide libraries because of the readily availability of amino acids and well known coupling methods. Some of these methods are the split-pool technique, pin technology, iterative deconvolution, physical isolation of active beads using the Selectide method, and encoded libraries.

Recently, attention has focused on the development of libraries of small organic molecules, due to their superior oral bioavailability, cell wall penetration, and duration of action. While progress has been made, many challenges remain. The complexity and diversity of many existing small molecule libraries are based on the attachment of multiple groups to a common scaffold resulting in moderate perturbations of the same structure. While such libraries are effective in optimizing the initial lead structure, greater diversity is needed to discover new bioactive compounds. In addition, most of the existing libraries are generated by a split-and-pool method. In the split and pool technique, individual compounds are combined in one vessel for washing and deprotection and then divided again into separate portions for the next coupling. In the case where polymers are produced in the split-and-pool technique, a statistical distribution of sequences is obtained. Although efficient, this method has several limitations including the requirement for additional encoding, generation of small quantities of final products, and inability to conduct multicomponent condensations.

A system and method for a parallel synthesis of a library of compounds comprising a plurality of plates having reaction wells which may be stacked to form a three-dimensional array has been described. One variation comprises several reaction well plates separated by one or more membrane sheets and stacked to form a three dimensional array having inlets for reagent delivery either from the top or the bottom of the array.

### BRIEF SUMMARY OF THE INVENTION

The present invention provides devices and methods for multiparallel synthesis and screening using three-dimensional arrays with a means for delivery of reagents along a minimum of three perpendicular axes. A plurality of reaction wells arranged in a three-dimensional array allow the introduction of a fluid or solid into the reaction wells along the x, y, and z axes of the array. The invention preferably uses a plurality of removable sealing elements each of which is positioned across or spans two sides of a reaction well when in a sealing position.

The present invention provides several benefits. It allows diversification of the library through reagent introduction or functionalization along the x, y, and z axes. Compounds can be cleaved without removing the beads from array. From 5-15 mg of individual library members may be produced and the multiparallel synthesis of greater than 1,000 individual compounds may be obtained. Further, the present invention enables positional encoding.

In one aspect, a device is provided that introduces a fluid or solid into a plurality of inlets at two or more adjacent faces or surfaces, or at two or more faces or surfaces that share a common edge or side, of the three-dimensional array. In another aspect, the device includes a plurality of removable sealing elements each of which is positioned across or spans two sides of a reaction well when in a sealing position. In still another aspect, the device includes a means for adding a fluid or solid to the reaction wells in a nonperpendicular direction relative to the plane of the reaction well layer or plate. In another aspect, a method includes introducing two or more fluids or solids into rows and columns of reaction wells, wherein at least three of the rows and columns are perpendicular to each other.

The present invention includes various non-limiting embodiments or modifications of the invention several of which are discussed in details below.

### BRIEF DESCRIPTION OF SEVERAL VIEWS OF THE DRAWINGS

FIG. 1 shows a matrix representation of molecular libraries.

FIG. 2 shows a reaction well of the device and a 2×2×2 and a 5×5×5 arrays for use in multiparallel synthesis.

FIG. 3 shows a 125 compound 3-dimensional multiparallel synthesizer.

FIG. 4 shows a 1000-compound array for use in multiparallel synthesis.

FIG. 5A-C show a top view of three different embodiments of the reaction well plate of the invention.

FIG. 6A-C show a side view of three different embodiments of the reaction well plate of the invention.

FIG. 7 shows a unit layer comprising an array of reaction wells each of which contains a solid-phase support and a 3-dimensional multiparallel synthesizer with connections to the reagent/solvent reservoirs.

FIG. 8 depicts the synthesis of a 125-member library of tripeptides.

### DETAILED DESCRIPTION OF THE INVENTION

DEVICES

The invention is directed to various embodiments of a device for multiparallel synthesis based on the three-dimensional arraying of interconnected reaction wells. The invention offers several useful features. For example, it provides positional encoding, improved efficiency compared to the traditional 96-well plate technique, and the techniques allows both solid-phase and solution-phase synthesis.

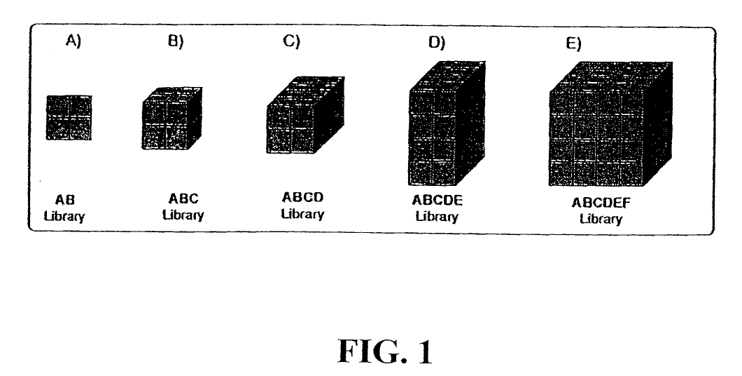

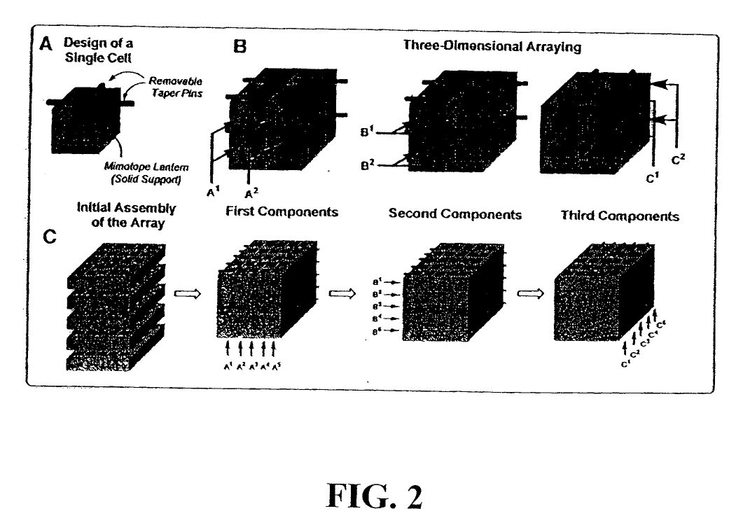

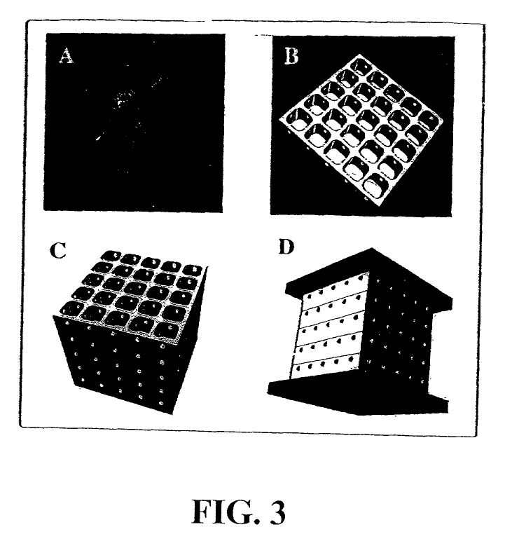

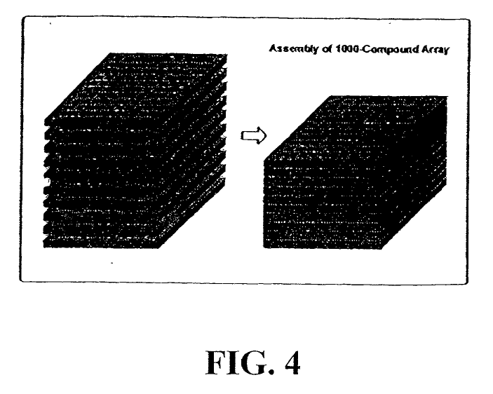

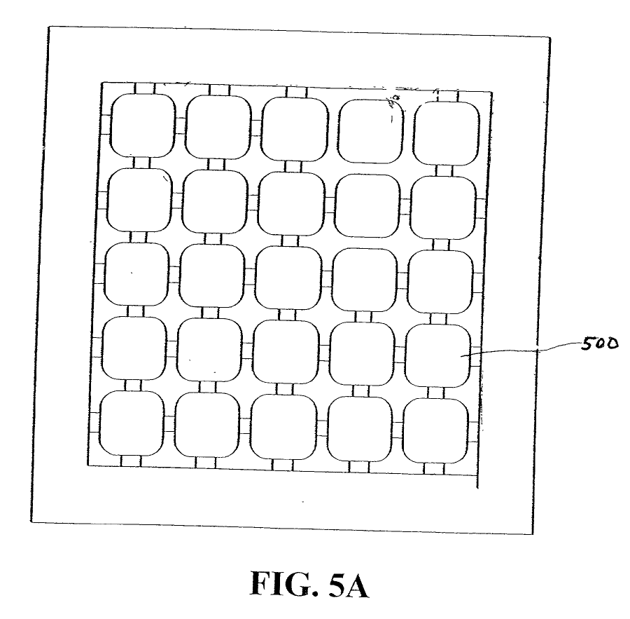

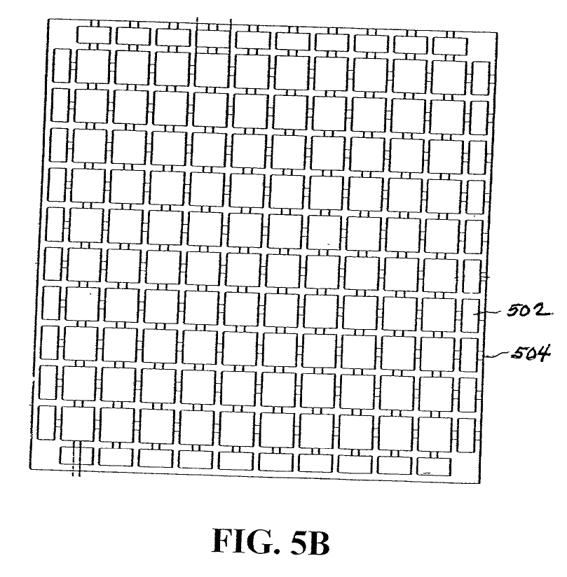

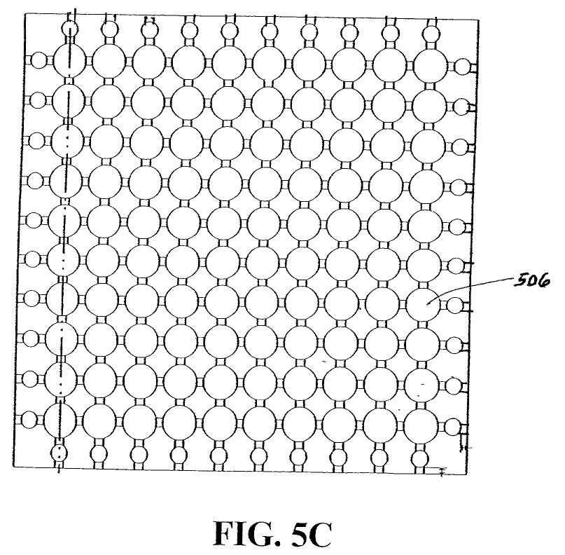

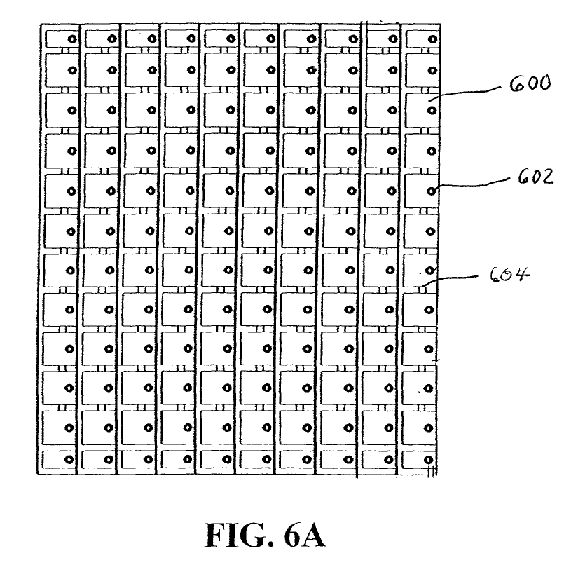

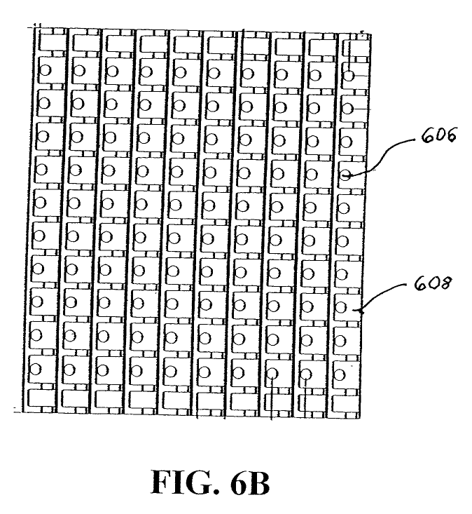

The device may include one or more arrays of vertically-stacked parallel multiple-well plates. FIG. 1 shows matrix representation of molecular libraries in the form of a single cubical reaction well. FIG. 2 shows an exemplary reaction well and a 2×2×2 and a 5×5×5 arrays for use in multiparallel synthesis. FIG. 3 shows a 125 compound 3-dimensional multiparallel synthesizer while FIG. 4 shows a 1000-compound array. FIG. 5A-C show a top view of three different embodiments of the reaction well plate of the invention. FIG. 5A-C illustrates plates that contain a plurality of reaction wells 500, 502, 506 and openings 504. FIG. 6A-B show a cross-sectional view that depict reaction well plates having a plurality of reaction wells 600, 608 and openings 602, 604, 606.

In a preferred embodiment of the invention, a device is provided that includes a plurality of reaction wells for receiving a fluid or solid, and a means for introducing two or more fluids or solids into at least two rows and at least one column of reaction wells, wherein a first row of reaction wells, a second row of reaction wells, and a column of reaction wells are perpendicular to each other. Thus, if a column (that is, a vertical or an upright arrangement) of reaction wells in a three-dimensional array corresponds to the z-axis, the first row and the second row (or vice versa) of reaction wells in the three-dimensional array correspond to the x and y axes, respectively. In another preferred embodiment of the invention, a three-dimensional array for multiparallel synthesis or screening includes a plurality of reaction wells for receiving a fluid or solid, and means for introducing the fluid or solid into a plurality of inlets at two or more adjacent faces or surfaces of the three-dimensional array.

In one embodiment, the invention provides a device for multiparallel synthesis or screening comprising a plurality of reaction wells each of which includes a plurality of reaction wells for receiving a fluid or solid, means for introducing the fluid or solid into the reaction wells, and a plurality of removable sealing elements each of which is positioned across or spans two sides of a reaction well when in a sealing position. In still another embodiment, the invention provides a device for multiparallel synthesis or screening comprising a plurality of reaction wells for receiving a fluid or solid, a means for introducing two or more fluids or solids into rows and columns of reaction wells, wherein at least three of the rows and columns are perpendicular to each other, and a plurality of removable sealing elements each of which is positioned across or spans two sides of a reaction well when in a sealing position.

In another embodiment, the invention provides a device for multiparallel synthesis or screening comprising a plurality of reaction wells for receiving a fluid or solid, a means for adding a fluid or solid to the reaction wells in a nonperpendicular direction relative to the plane of the reaction well layer or plate, and a plurality of removable sealing elements. The invention is also directed to an embodiment in which a device for multiparallel synthesis or screening includes a plurality of reaction wells for receiving a fluid or solid, means for adding a fluid or solid to the reaction wells in a nonperpendicular direction relative to a long axis of a layer or plate of reaction wells, and a plurality of removable sealing elements each of which is positioned across or spans two sides of a reaction well when in a sealing position.

Various configurations of the three-dimensional arrays of the invention are possible. The arrays may be used interconnectedly and may be driven by one or more pumps. Thus, the products or intermediates produced in a given array may be directed to another array which is directly or indirectly connected to the other array. The intervening arrays may be used for the washing steps or they may be used to introduce one or more reagents through one of the possible points or sides of entry. The functionalization, linking, or cleaving steps may be performed in one or more of the arrays.

The reaction wells receive and hold reagents, solvents, and/or one or more products produced in one or more reactions that occur in the reaction wells. In a preferred embodiment, two or more layers of reaction wells are stacked to form a three-dimensional array. The layers or plates of reaction wells may be held together by a top and bottom layers or plates which are connected, e.g., using screws to clamp tight the middle layers or plates of reaction wells, such that the fluids or solids in the reaction wells in each layer or plate are prevented from leaking. If desired, one or more layers of materials that are nonreactive or resistant to the reagents or solvents may be placed between the plates to further enhance the sealing between the layers of reaction wells.

Preferably, the reaction wells of the invention are made of materials such as stainless steel, aluminum, polypropylene, polyethylene, or PTFE, other suitable materials, as well as combinations of materials. The materials of which the reaction wells are made of are preferably resistant or nonreactive to the reagents or solvents used in the synthesis. The reaction wells comprising the array may number from less than 100, but they may also range from 1,000 reaction wells or more per array. FIG. 4 shows a 1000-compound array for use in multiparallel synthesis.

Preferably, a plurality of removable sealing elements are positioned across or span two sides of a reaction well or unit cell that houses a reaction well when each sealing element is in a sealing position. The sealing elements may include O-rings or tapered pins to enhance or facilitate the sealing or isolation of the reaction wells. In a preferred embodiment, sealing is achieved when the sealing elements are pushed or pulled individually or simultaneously (by installing one or more bars that connect several sealing elements, for example) such that the O-rings block the flow of fluid or solid when the O-rings lie within or coincide with the reaction well wall. When the sealing elements are pushed or pulled such that the O-rings are not coincident with the reaction well walls, one or more reagents or solvents may then be introduced into the reaction wells. The O-rings are preferably made of materials such as perfluoroelastomers sold under the name “KALREZ” (Dow Corning Chemical Company, Midland, Mich.), PTFE, or other materials that are resistant or non-reactive to the reagents or solvents used during the synthesis. In another aspect, the sealing elements include elements with varying diameters capable of allowing introduction of fluids or solids to the device.

In a preferred embodiment, the sealing elements include openings such as holes or grooves for reagent or solvent delivery. The distance between the openings in these sealing elements preferably coincides with the length of the sides of each reaction well. With these type of sealing elements, sealing is achieved when the sealing elements are pushed or pulled individually or simultaneously such that the reaction well wall material covers the holes or grooves in the sealing elements and no fluid leaks from the reaction well through the holes in the sealing elements.

The sealing elements are preferably in the form of cylinders, rods, tapered pins, or tubes. Preferably, the sealing elements are made of a material such as stainless steel, aluminum, PTFE, polypropylene, polyethylene, or a combination of materials such as polypropylene tubes that contains a stainless steel rod at the center. In a preferred embodiment, the sealing elements are in the form of stainless steel tapered pins.

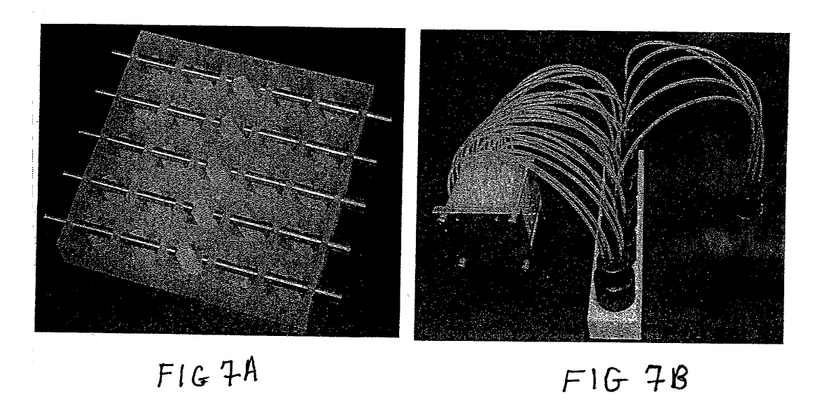

In a preferred embodiment, a plurality of reaction wells contains a solid-phase support. FIG. 7 shows an embodiment of a unit layer comprising an array of reactor wells each of which contains a solid-phase support and a 3-dimensional multiparallel synthesizer with connections to the reagent/solvent reservoirs.

The solid-phase support may be made of one or more materials such as polypropylene, polystyrene, polyamide, or polystyrene-poly(ethylene glycol) graft. The amount of solid-phase support placed in the reaction wells of an array depends on factors such as the desired amount of products to be synthesized, as well as the reaction well volume and the extent of swelling of the solid-phase support upon contact with a solvent or reagent. Commercially available solid-phase support such as resin beads and SynPhase™ lanterns may be used in various embodiments of the invention.

The solid-phase support may include beads or lanterns. The beads are preferably made of polypropylene or polyethylene. The choice of solid-phase support depends on various factors such as the desired amount of material to be synthesized (the loading capacity), compatibility of the chemistry intended for the library synthesis, and mode of attachment and cleavage of materials from the solid-phase support. Several beads may be placed in each unit well depending on factors such as the reaction well volume or the desired amount of product per reaction well. For example, a reaction well may include between about 5-10 beads per well. In a preferred embodiment, the solid-phase support does not contain any functionalities or linkers. The solid-phase support may be prefunctionalized and may contain one or more functionalities or linkers.

Solid-phase supports that may be used include materials such as polymers, metals, or glass. Examples of polymers include polypropylene, polystyrene, chloroacetyl polystyrene, carboxypolystyrene, polystyrene-CHO, and chloromethylated polystyrene. Resins such as those made of imidazole carbonate resin, polyacrylamide resin, benzhydrol resin, p-nitrophenyl carbonate resin, diphenylmethanol resin, trityl alcohol resin, hydroxymethyl resin, or triphenyl methanol polystyrene resin, or their various combinations may also be used.

The solid-phase supports may be functionalized with one or more chemically reactive groups which are used to attach a linker to a solid-phase support. Examples of these chemically reactive groups include, but are not limited to, isocyanates, carboxylic acids, esters, amides, alcohols, isothiocyanate, amines, and halomethyl groups.

Preferably, non-functionalized solid-phase supports are used during the parallel synthesis. Using non-functionalized solid-phase supports offers the advantages of flexibility and convenience because it obviates the need for prefunctionalizing the solid-phase support before putting them into the reaction wells. Various non-functionalized solid-phase supports are commercially available such as certain types of SynPhase™ Lanterns. Preferably, the non-functionalized solid-phase supports include polypropylene, polyethylene, or PTFE.

Different types of linkers may be used in the various embodiments of the invention. A linker covalently attaches molecules to the solid-phase support. The choice of a particular linker depends on factors such as the particular product or intermediate to be synthesized and the stability of a linker. Different types of linkers are known in the art and they are preferably attached to the solid-phase supports using standard solid-phase chemistry techniques. Preferably, the linkers are those based or adapted from protecting group chemistry.

Examples of linkers that may be used in the various embodiments of the invention include acid labile linkers, nucleophile labile linkers, safety-catch linkers, traceless linkers, and fluoride labile linkers. Photo-labile linkers may also be used in the various embodiments of the invention. An advantage of nucleophile labile linkers is that it can be used to introduce a moiety or functional group during the cleavage step. Safety-catch linkers allows cleavage of an activated linker using mild conditions. Photo-labile linkers can be used under mild conditions and the process can be selective.

Examples of linkers that may be used in the various embodiments of the invention include, but are not limited to, rink amide linkers, hydroxymethylphenoxy linker (HMP linker), backbone amide linker, trityl alcohol linker, disulfide linker, sulfoester linker, benzylhydryl or benzylamide linker, ortho-nitrobenzyl-based linker, nitroveratryoxycarbonyl-based linkers, and phenacyl based linkers.

Cleavage of the synthesis products can be performed using techniques known in the art. In one embodiment, a product is cleaved from the solid-phase support via an intramolecular reaction that removes the compound from the solid-phase support without leaving any trace of the site of attachment. See DeWitt, et al., Proc. Natl. Acad. Sci. U.S.A., 1993, 90: 6909-6913, which is incorporated herein in its entirety.

The following examples illustrate the use of some of the above linkers. Rink amide linkers, which are commercially available under the name SynPhase™, can be used with activated carboxylic acids which cleave to form primary carboxyamides. Rink amide linkers can also be loaded with sulfonyl chorided to produce primary sulfonamides. When using rink amide linker, cleavage is normally performed using 20% TFA/DCM. Solid-phase supports with HMP linker can be used to link carboxylic acids, phenols, and amines. In this case, cleavage via acidolysis produces the original functional group. The carboxylic acids can be coupled using N,N′-diisopropylcarbodiimide (DIC)/N,N-dimethylaminopyridine through imidate or Mitsunobu chemistry. With HMP linker, cleavage is normally performed with about 20% or higher concentrations of trifluoroacetic acid (TFA)/dichloromethane (DCM). Trityl alcohol linker can be used to link carboxylic acids, alcohols, phenols, and amines. Cleavage via acidolysis produces the original functional group. Hyperlabile linker links phenols, amines, and carboxylic acids. Cleavage by acidolysis recovers the original functional group.

Following the cleavage step, the cleaved compounds may be analyzed or characterized using one or more analytical techniques such as mass spectrometry, liquid chromatography, NMR, or a combination of techniques such as LC-UV/MS. On-site or on-bead analysis may be performed using techniques such as magic angle spinning NMR or FT-IR spectrometry. The applications of one or more of these techniques in solid-phase synthesis have been described in several publications including, for example, Chu et al., J. Org. Chem., 58: 648-652 (1993); Fitch et al., J. Org. Chem., 59: 7955-7956 (1994); Gao et al., J. Med. Chem., 39: 1949-1955 (1996); P. A. Keifer, J. Org. Chem., 61: 1558-1559 (1996); Metzger et al., Angew. Chem. Int. Ed., 32: 894-896 (1993); Stevanovich and Jung, Anal. Biochem., 212: 212-220 (1993); and Youngquist et al., Rapid Commun. Mass Spectrom., 8: 77-81 (1994), all of which are incorporated herein in their entirety.

Various types of solid-phase chemistries for the combinatorial synthesis of non-oligomeric small molecules are well-known in the art, and they may be used in conjunction with the various embodiments of the invention. Examples of these solid-phase techniques include, but are not limited to, those described in Gordon et al., J. Med. Chem., 37: 1385 (1994), Patel and Gordon, Drug Disc. Today, 4: 134-144 (1996); Fruchtel and Jung, Agnew. Chem., 35:17-41 (1996); Thompson and Ellman, Chem. Rev., 96: 555-600 (1996); Bunin and Ellman, J. Am. Chem. Soc., 114:10997 (1992); Gallop et al., U.S. Pat. No. 5,525,734, Bunin et al., Proc. Natl. Acad. Sci. U.S.A., 91: 4708 (1994); Plunkett and Ellman, J. Am. Chem. Soc., 117: 3306 (1995); Hobbs DeWitt et al, Proc. Natl. Acad. Sci. U.S.A., 90: 6909 (1993); Murphy et al., J. Am. Chem. Soc., 117: 7029 (1995); Holmes et al, J. Org. Chem., 60: 7328 (1995); Holmes, U.S. Pat. No. 5,549,974; Gordon and Steel, Bioorg. Med. Chem. Lett., 5: 47 (1995); Patek et al, Tetrahedron Lett. 36: 2227 (1995); Szardenings et al, Tetrahedron, 53: 6573 (1997); Beebe et al., J. Am. Chem. Soc., 114: 10061 (1992); Moon et al, J. Org. Chem., 57: 6088 (1992); Pei and Moos, Tetrahedron Lett., 35: 5825 (1994); and and Maclean, Proc. Natl. Acad. Sci. U.S.A., 94: 2805 (1997), each of which is incorporated herein in its entirety.

The invention permits positional or spatial encoding. Thus, there is no need for additional complicated and time-consuming steps to identify the structure of library members, such as electronic encoding, graphical encoding, chemical encoding, spectrometric encoding, or deconvolution techniques. The compounds can be identified simply from their particular positions in the array.

Preferably, the smallest side of a reaction well is at least about 10 mm. In a preferred embodiment, each solid-phase support has a loading capacity of at least about 30 μmol. Preferably, the synthesis produces at least about 1 mg of a reaction product, more preferably at least about 3-4 mg of a reaction product.

In a highly preferred embodiment of the invention, the fluids include small molecule reactants. The chemistry of several non-peptide libraries have been described in the art. See, for example, Cho et al., Science, 261: 1303-1305; DeWitt et al., Proc. Natl. Acad. Sci. U.S.A., 90: 6909-6913; Simon et al, Proc. Natl. Acad. Sci. U.S.A., 89: 9367-9371; Zuckermann et al., J. Amer. Chem. Soc., 114: 10646-10647; and Zuckermann et al., J. Med. Chem., 37: 2678-2685, all of which are incorporated herein in their entirety. The chemistry and methods described in these references may be used in conjunction with the devices and methods of the invention. In one embodiment, the fluid includes one or more biomolecule.

Methods

The present invention includes a method for synthesizing or screening an array of compounds. Preferably, the method includes introducing two or more fluids or solids into two or more rows of reaction wells and at least one column of reaction wells, wherein a first row of the reaction wells lies perpendicular to a second row of reaction wells, and the at least one column of reaction wells lies perpendicular to each of the first row and the second row of reaction wells, and inserting a plurality of removable sealing elements. Preferably, the method includes introducing a fluid or solid into a plurality of inlets at two or more adjacent faces or surfaces of the three-dimensional array, and inserting a plurality of removable sealing elements.

FIG. 2 shows a synthesis protocol using a 2×2×2 array as a representative example. The process begins with the removal of four pins from one side of the cube. The additions of the two components A₁ and A₂ to the first two layers of the array are carried out simultaneously. The pins inserted in a perpendicular direction provide the necessary sealing and avoid the undesired mixing between reactants A₁ and A₂. Following the washing cycle, the process is repeated using the next set of reagents B₁ and B₂ by changing the direction of reagent addition by 90°. Following another washing cycle, the four pins are reinserted as shown in FIG. 2B. This operation allows the last two components C₁ and C₂ to be delivered on an adjacent face or surface of the array, thus completing the synthesis process. At the end of the synthesis, the layers of the array can be physically separated to allow the removal of the material from the reactor wells either prior to or following the solid-phase cleavage step. Although compact in size, the device of the invention allows the preparation of milligram quantities of final products, which is sufficient for performing hundreds of biological assays.

Extension of the method above to a 5×5×5 array is shown in FIG. 3. This array is capable of delivering 125 spatially localized and positionally encoded components. FIG. 7 shows a photograph of a 5×5×5 array. This system allows coupling steps to be conducted within a given layer of the array without cross-contamination. Teflon gaskets may be used to achieve fluid-tight seals between the layers. Each well is equipped with a single 12.5 mm SynPhase lantern. Based on the typical loading capacity of 35 pmol/lantern, the synthesis can produce 5-15 mg of individual library members. FIG. 7A shows a representative 25-well polypropylene plate. Each well has four openings allowing for insertion of tapered stainless steel pins of the appropriate diameter to achieve fluid-tight sealing when the pin is inserted to block the appropriate openings. Removal of the pin connects the adjacent reactors, thus allowing the addition of reagents.

Preferably, the method includes introducing a fluid or solid into reaction wells of the array, and inserting a plurality of removable sealing elements each of which is positioned across or spans two sides of a reaction well when in a sealing position. In still another embodiment, the method includes adding a fluid or solid into rows and columns of inlets of reaction wells, wherein at least three of the rows and columns are perpendicular to each other, and inserting a plurality of removable sealing elements each of which is positioned across or spans two sides of a reaction well when in a sealing position.

In another embodiment, the method for synthesizing or screening an array of compounds includes adding a fluid or solid in a nonperpendicular direction relative to the plane of at least one of two or more layers or plates of reaction wells, and inserting sealing elements into the reaction wells following the addition of the fluid or solid. In another embodiment, the method for synthesizing or screening an array of compounds comprising adding a first fluid or first solid to a first set of two or more layers or plates of a device comprising reaction wells in a nonperpendicular direction relative to the plane of at least one of two or more layers or plates of reaction wells, inserting sealing elements into the reaction wells, and adding a second fluid or second solid to a second set of two or more layers or plates of the device.

In a further embodiment, the method for synthesizing or screening an array of compounds includes adding a first fluid or first solid to a first two layers of a device comprising reaction wells, inserting a plurality of sealing elements into the reaction wells following the addition of a first fluid or first solid to the first two layers of the device comprising reaction wells, and adding a second fluid or second solid in a direction perpendicular to a direction of addition of the first fluid or first solid, wherein points of entry of the fluid or solid through the inlets define a line of direction of addition.

In a preferred embodiment, the method of the invention includes performing at least one washing step after the addition of the fluids or solids. Preferably, a washing step is performed after each addition of a fluid or solid using suitable solvents such as water, organic solvents, aqueous or organic solutions, or combinations of suitable solvents.

In a preferred embodiment of the invention, the addition of the fluid or solid is performed at least three times. The fluid or solid may be added using delivery systems or devices such as syringes. In one embodiment, the fluid or solid is added using tubes. Preferably, the fluid or solid are added with the aid of a pump. Different types of pumps such as syringe pumps, peristaltic pumps, piston metering pumps, and diaphragm pumps are commercially available. In a preferred embodiment, the pump is a syringe pump. The pumps suitable for use in the various embodiments of the invention are those having components that are preferably resistant to the solvent or reagents used in the synthesis. Preferably, the pumps can be operated under a wide range of solution temperatures.

Inert gas such as argon or nitrogen may be used to allow pressurized delivery or transfer of a reagent. For example, a pressurized inert gas such as argon or nitrogen may be applied to a tube to force a fluid from a reservoir into the array. The reservoirs themselves may be subjected to pressure to allow or facilitate the transfer of a reagent or solvent from a reservoir into the reaction wells of the array. In a preferred embodiment, one or more valves, which are electronically controlled, such as solenoid valves with a plurality of ports, are used in the reagent or solvent transfer.

### EXAMPLE

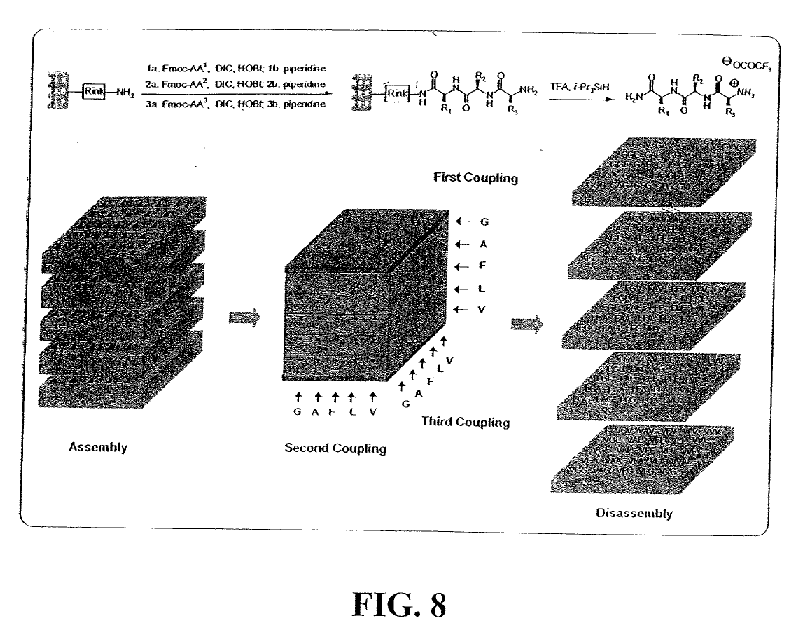

A synthesis of a 125-peptide library using the system shown in FIG. 8. The library was generated by randomization of the tripeptide at each position with five amino acids Gly, Ala, Phe, Val, and Leu. A conventional 9-fluorenylmethoxycarbonyl (Fmoc)-solid-phase peptide synthesis using N-hydroxybenzotriazole (monohydrate) (HOBt)/DIC coupling protocol using commercially available SynPhase D-series PA lanterns with Rink linker. The three-dimensional arraying of the five amino acids is schematically illustrated in FIG. 8. The synthesis was carried out within the expected 10-hour period. Examination of 25 peptides, randomly selected from the final array using ¹H-NMR spectroscopy and HPLC-MS confirmed their identities and high chemical purity. This particular example of parallel synthesis can be extended to the preparation of a 1,000-compound (or greater) three-dimensional arrays equipped with readily available solid-phase supports such as 500-600 um polystyrene beads.

1. A device for multiparallel synthesis or screening comprising
(a) a plurality of reaction wells for receiving a fluid or solid,
(b) a means for introducing the fluid or solid into one or more reaction wells of said plurality, and
(c) a plurality of removable sealing elements each of which is positioned across, or spans two sides of, a reaction well when in a sealing position.

2. A device for multiparallel synthesis or screening comprising
(a) a plurality of reaction wells that form a three dimensional array for receiving a fluid or solid, and
(b) a means for introducing the fluid or solid into a plurality of inlets at two or more adjacent faces or surfaces of the three-dimensional array.

3. A device for multiparallel synthesis or screening comprising
(a) a plurality of reaction wells for receiving a fluid or solid,
(b) a means for introducing a fluid or solid into a first row of reaction wells,
(c) a means for introducing a fluid or solid into a second row of reaction wells which is perpendicular to the first row of reaction wells, and
(d) a means for introducing a fluid or solid into a column of reaction wells which is perpendicular to the first row and the second row of reaction wells.

4. A device according to claim 1, wherein the sealing elements are tapered, cylindrical, or rod-shaped.

5. A device according to claim 1, wherein the sealing elements are tapered pins.

6. A device according to claim 1, wherein the sealing elements have varying diameters.

7. A device according to claim 1, wherein the fluid or solid is introduced through one or more openings in the sealing elements.

8. A device according to claims 1, 2, or 3, wherein the reaction wells further comprise solid-phase supports that do not contain preformed functionalities or linkers.

9. A device according to claims 1, 2, or 3, wherein the reaction wells are made of polypropylene, polyethylene, PTFE, or stainless steel.

10. A device according to claims 1, 2, or 3 wherein the smallest side of a reaction well is at least about 10 mm.

11. A device according to claims 1, 2, or 3 wherein at least about 1 mg of a reaction product is produced.

12. A device according to claims 1, 2, or 3, wherein two or more layers of said reaction wells are stacked to form a three-dimensional array.

13. A device according to claim 2, wherein the three-dimensional array is connected to one or more three-dimensional arrays.

14. A method for synthesizing or screening compounds comprising
(a) introducing a fluid or solid into a first row of reaction wells,
(b) introducing a fluid or solid into a second row of reaction wells that lie perpendicular to the first row of reaction wells,
(c) introducing a fluid or solid into a column of reaction wells that lie perpendicular to each of the first row and the second row of reaction wells, and
(d) inserting a plurality of removable sealing elements.

15. A method for synthesizing or screening compounds comprising
(a) introducing a fluid or solid into a plurality of inlets at two or more adjacent faces or surfaces of a three-dimensional array that comprises a plurality of reaction wells, and
(b) inserting a plurality of removable sealing elements.

16. A method according to claims 14 or 15, wherein each of the plurality of removable sealing elements is positioned across, or spans two sides of, a reaction well when in a sealing position.

17. A method according to claims 14 or 15 further comprising performing one or more washing steps after introducing one or more fluids or solids.

18. A method according to claims 14 or 15, wherein the fluid or solid is added using syringes or tubes.

19. A method according to claims 14 or 15, wherein at least one milligram of a reaction product is produced.

20. A method according to claims 14 or 15, wherein the fluid or solid is added using a pump.

21. A method according to claims 14 or 15 further comprising using a solid-phase support made of polypropylene, polyethylene, perfluoroelastomer, or PTFE.

22. A method for synthesizing an array of compounds comprising using the device of claims 1, 2, or 3 to produce at least one milligram of a reaction product.

23. A method according to claims 14 or 15, wherein the fluid or solid comprises small molecule reactants.

24. A method according to claims 14 or 15, wherein functionalization, linking, or cleavage is performed in the reaction wells.
